(19) United States
(12) Patent Application Publication
(10) Pub. No.: US 2003/0195217 A1
(43) Pub. Date: Oct. 16, 2003

(54) 3,9-Diazabicyclo[3.3.1]nonane derivatives with analgesic activity

(76) Inventors: Giorgio Cignarella, Milano (IT); Gerard Aime Pinna, Sassari (IT)

Correspondence Address: YOUNG & THOMPSON, 745 SOUTH 23RD STREET 2ND FLOOR, ARLINGTON, VA, 22202

(21) Appl. No.: 10/221,209

(22) PCT Filed: Feb. 13, 2001
(86) PCT No.: PCT/EP01/01541

(30) Foreign Application Priority Data
Feb. 18, 2000 (IT) MI2000A000293

(51) Int. Cl. A61K 31/498; C07D 487/04

(52) U.S. Cl. 514/249; 544/349

(57) ABSTRACT

Compounds of formula (I) wherein R and R₁, which are different from each other, are a straight or branched C₂-C₈ acyl group, have analgesic activity.

The present invention relates to 3,9-diazabicyclo[3.3.1]nonane derivatives, the use thereof for the preparation of medicaments with central analgesic activity and pharmaceutical compositions containing them.

In particular, the invention relates to compounds of general formula (I)

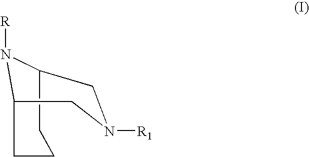

wherein

R and R₁, which are different from each other, are a straight or branched C₂-C₈ acyl group;

a group of formula

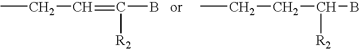

wherein:

B is a C₆-C₁₀ aryl group, optionally substituted at the ortho-, meta- or para-positions with one or more substituents, which are the same or different, selected from the group consisting of C₁-C₃ alkoxy, C₁-C₂ halo alkyl, C₁-C₃ alkyl, halogens, carboxy, cyano, nitro, CONHR₃; a C₅-C₇ cycloalkyl group, a 5 or 6 membered heterocyclic aromatic group, optionally benzofused, having at least one heteroatom selected from nitrogen, oxygen, sulfur; said heterocyclic group optionally having one or more substituents as described above for the aryl group;

R₂ is hydrogen, C₁-C₄ alkyl, C₅-C₇ cycloalkyl or a phenyl group optionally substituted as indicated above,

and the pharmaceutically acceptable salts thereof.

Examples of C₁-C₈ acyl groups are acetyl, propionyl, isopropionyl, butyryl, isobutiryl, valeryl, isovaleryl, pivaloyl, caproyl.

Examples of heterocyclic groups are pyrrole, furan, thiophene, imidazole, oxazole, thiazole, pyridine, pyrimidine, pyridazine, pyrazine, benzothienyl.

Examples of pharmaceutically acceptable salts are those with halohydric acids, such as hydrochloric acid, hydrobromic acid; mineral acids, such as sulfuric and phosphoric acids; organic acids, such as acetic, propionic, succinic, glutaric, benzoic, salicylic acids. Any carboxylic groups can be in the salified form with alkali or alkaline-earth metal bases, such as sodium, potassium, calcium, magnesium; bases of non toxic metals; non toxic organic amines.

Preferred are compounds of formula (I) wherein R or R₁ are an acyl group as defined above or a group of formula

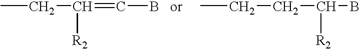

and B is a phenyl group, optionally substituted, as defined above, a naphthyl or a heterocyclic group.

Also preferred are compounds of formula (I) wherein R₁ is an acyl group as defined above and R is the group of formula

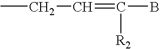

3,8-Diazabicyclo[3.2.1.]octane derivatives with analgesic activity are disclosed in EP 0 746 560.

It has now been found that the compounds of formula (I) have central analgesic activity comparable to that of morphine and higher than that of 3,8-diazabicyclo[3.2.1.]octane, are “substantially free” from withdrawal symptoms and less liable than morphine to induce tolerance or physical dependence after chronic treatment.

“Substantially free” herein means an activity 3 to 20 times lower than that of morphine in the mouse jumping test, after chronic administration three times a day for 7 consecutive days of analgesically equipotent dosages.

The present invention also relates to the compounds of general formula (I) as agents with central analgesic activity.

A further object of the present invention are the processes for the preparation of said compounds.

Still a further object of the present invention is the use of the compounds of formula (I) for the preparation of a medicament useful to induce analgesia on central nervous system in a mammal, particularly in humans, requiring such treatment.

Still a further object of the invention are pharmaceutical compositions containing a therapeutically effective amount of at least one compound of formula (I) in mixture with conventional carriers and excipients.

The compounds of the invention can be prepared by reaction of intermediates of formula (IIa) or (IIb)

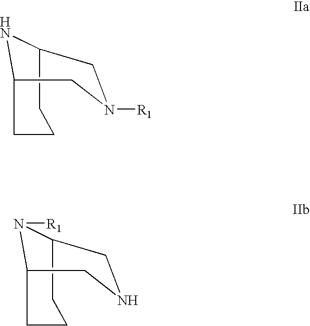

wherein R′ is a straight or branched C₂-C₈ acyl group

with a compound of formula

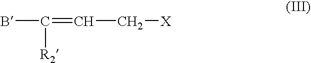

wherein R₂′ and B′ have the same meanings as R₂ and B or are groups which can be transformed into R₂ and B, and X is a leaving group, for example a halogen atom, mesyl, tosyl and the like.

The reactions described above are carried out according to conventional techniques known to those skilled in the art. Reagents are usually present in stoichiometric or slightly different ratios, depending on the reactivity of the specific reagent.

The acylation of the nitrogen at 3 or at 9 is usually carried out with acid chlorides in an inert reaction medium, such as an open or closed chain ether, a ketone, an optionally halogenated hydrocarbon, preferably in the presence of a proton acceptor, such as a tertiary amine. Alternatively, the acylating agent can be a carboxylic acid anhydride.

The intermediates of formulae (IIa) and (IIb) can be obtained. by acylation, according to conventional methods, of a compound of formula (IVa) or (IVb)

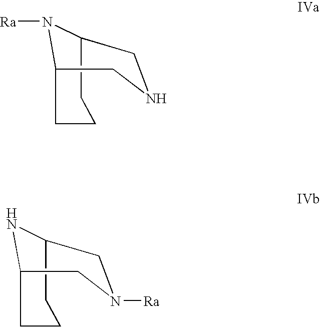

wherein Ra is an amino-protecting group, and subsequent removal of the protective group. Compound of formula (IVa) in which Ra is benzyl is known from Gazzetta Chimica Italiana, 1963, 226-227, and can be prepared according to the following scheme 1

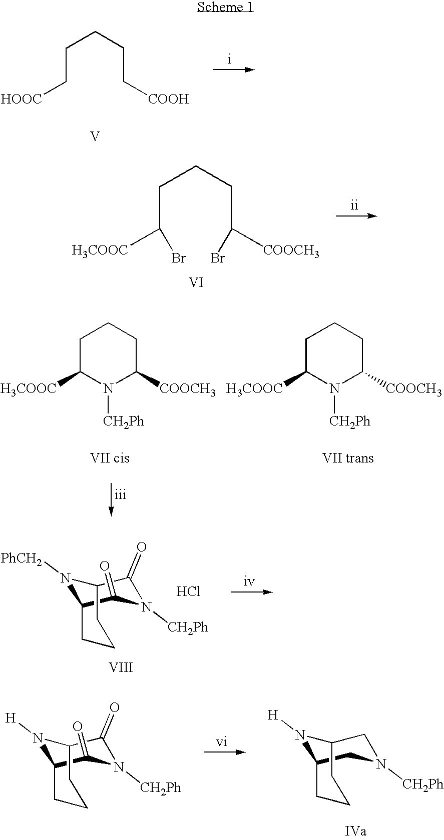

Meso-dimethyl-α,α-dibromopimelate (VI) obtained by bromination of pimelic acid (V), is condensed with benzylamine in benzene under reflux to give N-benzyl-2,6-dicarbomethoxy-piperidine (VII) as cis and trans isomeric mixture, which is reacted with benzylamine in xylene under reflux for 18 hours and then, after evaporation of the solvent, for a further 4 hours a 160-170° C.

The resulting compound (VIII) is recovered as hydrochloride from the reaction product by dissolution in ethanol and precipitation with HCl, then is hydrogenolysed to give the compound (IX) which is reduced with metal hydrides such as LiAlH₄, to yield compound (IVa).

Compounds (IVb) can be obtained from compounds (IVa) through thermal rearrangement, analogously to what published for the homologous diazabicyclooctanes (Tetrahedron, 1963, 9, 143-148).

Intermediates of formula (III) are known or can be prepared with known methods, for example by reducing suitable arylacryl acids or esters thereof with metal hydrides and subsequently transforming the resulting alcohol into halide, with conventional methods, according to Scheme 2 reported in the following, concerning compounds (III) in which B is optionally substituted phenyl and R₂ is hydrogen. Other compounds of formula (III) can be obtained with similar methods.

In Scheme, R₃ represents the substituents listed for the aryl group R₂.

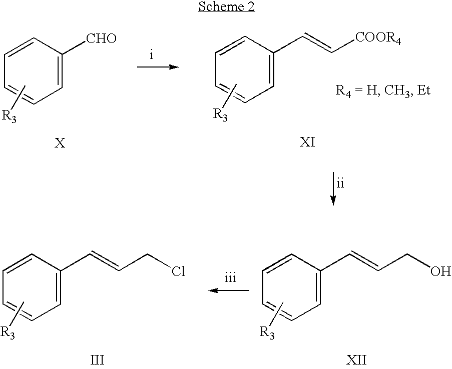

Compounds (I) and the salts thereof with pharmaceutically acceptable acids can be advantageously used as active principles in medicaments having central analgesic activity, as well as poor liability to induce tolerance and withdrawal symptoms which are the most serious restrictions to the use of morphine.

For the envisaged therapeutical uses, compounds (I) or the salts thereof will be formulated in a therapeutically effective amount in suitable pharmaceutical formulations according to conventional techniques and excipients, such as those described in “Remington's Pharmaceutical Sciences Handbook” XVII Ed. Mack Pub., N.Y., USA.

Examples of pharmaceutical compositions are tablets, capsules, granulates, powders soluble, drops, elixirs, syrups, injectable forms, suppositories.

The dosages and posology will be defined by the physician depending on the severity of the disease, the conditions of the patient and any possible interactions with other medicaments.

The following examples further illustrate the invention.

Preparation 1

3-Propionyl-3.9-diazabicyclo[3.3.1]nonane

9-Propionyl-3,9-diazabicyclo[3.3.1.]nonane (IVa) (0.83 g, 4.56 mmol) obtained according to Gazzetta Chimica Italiana 1963, 226-227 was heated at 150° C. for 2 hours. The crude product was chromatographed (silica gel) eluting with CHCl₃—CH₃OH/8:2.

The title product was recovered from the fraction with R_{f} 0.29 as oil, b.p. 125-130° C./0.4 mmHg. IR (film, cm⁻¹) v: 1630 (C═O), 2920 (NH); ¹H-NMR (CDCl₃) δH: 1.16 (t, 3H), 1.50-1.70 (m, 2H), 1.80-2.20 (m, 4H), 2.35 (q, 2H), 3.15 (dd, 1H), 3.33 (br s, 2H), 3.65 (dd, 1H), 3.88 (d, 1H), 4.79 (br s, 1H exch. with D₂O). ¹³C-NMR (CDCl₃) δc: 9.05 (CH3), 18.24, 26.64, 29.48, 29.49, 45.08 and 49.22 (CH2x6), 46.53 and 46.61 (CHx2), 172.58 (C═O) from DEFT (135° C.) and HETCOR.

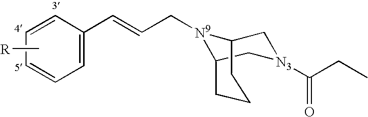

| | | Yield | m.p. | Formula | IR^{C} | ¹H-NMR |
|---|---|---|---|---|---|---|
| Ex. | R | % | ° C. | (Analysis^{b}) | ν cm⁻¹ | δ ppm |
| 8 | H | 36 | oil | C₁₉H₂₆N₂O | 1525, 1635 | 1.19 (t, 3H); 1.46-1.66 (m, 2H); 1.72-2.20 (m, 4H); 2.21- |
| | | | | (C, H, N) | | 2.40 (m, 2H); 2.92 (br s, 2H); 3.18 (dd, 1H); 3.50-3.80 |
| | | | | | | (m, 4H); 4.40 (d, 1H); 6.20-6.30 (dt, 1H); 6.60 (d, 1H); |
| | | | | | | 7.20-7.40 (m, 5H). |
| 9 | 4′-NO₂ | 22 | oil | C₁₉H₂₅N₃O₃ | 1360, 1515 | 1.19 (t, 3H); 1.47-1.70 (m, 2H); 1.72-2.20 (m, 4H); 2.21-2.40 |
| | | | | (C, H, N) | 1630 | (m, 2H); 3.01 (br s, 2H); 3.50-3.70 (m, 5H); 4.37 (d, 1H); |
| | | | | | | 6.30-6.40 (dt, 1H); 6.60 (d, 1H); 7.50 (d, 1H); 8.20 (d, 2H). |
| 10 | 3′-Cl | 27 | oil | C₁₉H₂₅ClN₂O | 1630 | 1.17 (t, 3H); 1.40-1.60 (m, 2H); 1.70-2.20 (m, 4H); 2.30- |
| | | | | (C, H, N) | | 2.50 (m, 2H); 2.98 (br s, 2H); 3.10 (dd, 1H); 3.40-3.60 (m, |
| | | | | | | 4H); 4.40 (d, 1H); 6.20-6.40 (dt, 1H); 6.45 (d, 1H); 7.01- |
| | | | | | | 7.40 (m, 4H). |
| 11 | 3′,4′-Cl₂ | 36 | oil | C₁₉H₂₄Cl₂N₂O | 1635 | 1.17 (t, 3H); 1.40-1.60 (m, 2H); 1.70-2.10 (m, 4H); 2.20- |
| | | | | (C, H, N) | | 2.40 (m, 2H); 2.89 (br s, 2H); 3.40-3.60 (m, 5H); 4.20 (d, |
| | | | | | | 1H); 6.20-6.30 (dt, 1H); 6.40 (d, 1H); 7.10-7.20 (m, 1H); |
| | | | | | | 7.30-7.50 (m, 2H). |
| 12 | 3′-NO₂, 4′-Cl | 60 | oil | C₁₉H₂₄ClN₃O₃ | 1330, 1520 | 1.19 (t, 3H); 1.42-1.62 (m, 2H); 1.70-2.20 (m, 4H); 2.20- |
| | | | | (C, H, N) | 1630 | 2.40 (m, 2H); 2.92 (br s, 2H); 3.15 (dd, 1H); 3.40-3.60 (m, |
| | | | | | | 4H); 4.40 (d, 1H); 6.20-6.40 (dt, 1H); 6.52 (d, 1H); 7.40- |
| | | | | | | 7.60 (m, 2H); 7.80 (s, 1H). |
| 13 | 2′-NO₂, 5′-Cl | 25 | 130 (dec)^{a} | C₁₉H₂₄ClN₃O₃.HCl | 1340, 1520 | 1.17 (t, 3H); 1.42-1.65 (m, 2H); 1.70-2.20 (m, 4H); 2.37 |
| | | | | (C, H, N) | 1635 | (q, 2H); 2.93 (br s, 2H); 3.12 (dd, 1H); 3.50-3.75 (m, 4H); |
| | | | | | | 4.40 (d, 1H); 6.15-6.30 (dt, 1H); 7.01 (d, 1H); 7.30 (dd, |
| | | | | | | 1H); 7.56 (d, 1H); 7.92 (d, 1H). |
| 14 | 2′-Cl, 5′-NO₂ | 30 | 245^{a} | C₁₉H₂₄ClN₃O₃.HCl | 1340, 1520 | 1.17 (t, 3H); 1.48-1.68 (m, 2H); 1.72-2.18 (m, 4H); 2.34 |
| | | | | (C, H, N) | 1560, 1635 | (dq, 2H); 2.93 (br s, 2H); 3.15 (dd, 1H); 3.42-3.78 (m, |
| | | | | | | 4H); 4.40 (d, 1H); 6.30-6.50 (dt, 1H); 7.01 (d, 1H); 7.65 |
| | | | | | | (d, 1H); 8.05 (dd, 1H); 8.42 (d, 1H). |

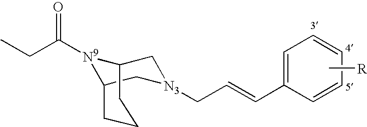

| | | Yield | m.p. | Formula | IR^{C} | ¹H-NMR |
|---|---|---|---|---|---|---|
| Ex. | R | % | ° C. | (Analysis^{b}) | ν cm⁻¹ | δ ppm |
| 1 | H | 72 | oil | C₁₉H₂₆N₂O | 1635 | 1.16 (t, 3H); 1.40-1.60 (m, 1H); 1.70-1.95 (m, 4H); |
| | | | | (C, H, N) | | 2.20-2.40 (m, 4H); 2.70-3.15 (m, 5H); 3.88 (br s, 1H); |
| | | | | | | 4.70 (br, s, 1H); 6.20-6.40 (dt, 1H); 6.50 (d, 1H); 7.20- |
| | | | | | | 7.40 (m, 5H). |
| 2 | 4′-NO₂ | 34 | oil | C₁₉H₂₅N₃O₃ | 1350-1510 | 1.17 (t, 3H); 1.50-1.70 (m, 1H); 1.70-1.92 (m, 4H); |
| | | | | (C, H, N) | 1620 | 2.20-2.40 (m, 4H); 2.65-3.20 (m, 5H); 3.95 (br s, 1H); 4.73 |
| | | | | | | (br, s, 1H); 6.40-6.60 (m, 2H); 7.55 (d, 2H); 8.20 (d, 2H). |
| 3 | 3′-Cl | 64 | oil | C₁₉H₂₅ClN₂O | 1640 | 1.18 (t, 3H); 1.40-1.60 (m, 1H); 1.70-1.93 (m, 4H); 2.20- |
| | | | | (C, H, N) | | 2.40 (m, 4H); 2.80-3.10 (m, 5H); 3.88 (br s, 1H); 4.68 (br, |
| | | | | | | s, 1H); 6.10-6.30 (dt, 1H); 6.50 (d, 1H); 7.20-7.30 (m, 4H). |
| 4 | 3′4′-Cl₂ | 72 | oil | C₁₉H₂₄Cl₂N₂O | 1635 | 1.11 (t, 3H); 1.42-1.63 (m, 1H); 1.70-1.90 (m, 4H); 2.20- |
| | | | | (C, H, N) | | 2.40 (m, 4H); 2.80-3.10 (m, 5H); 4.05 (br s, 1H); 4.65 (br, |
| | | | | | | s, 1H); 6.10-6.30 (dt, 1H); 6.40 (d, 1H); 7.10-7.50 (m, 3H). |
| 5 | 3′-NO₂, 4′-Cl | 76 | oil | C₁₉H₂₄ClN₃O₃ | 1335, 1524 | 1.15 (t, 3H); 1.50-1.70 (m, 1H); 1.75-1.95(m,4H); 2.22- |
| | | | | (C, H, N) | 1630 | 2.42 (m, 4H); 2.85-3.25 (m, 5H); 3.89 (br s, 1H); 4.73 (br, |
| | | | | | | s, 1H); 6.15-6.24 (dt, 1H); 6.40-6.50 (m, 2H); 7.40 (br s, |
| | | | | | | 2H); 7.80 (s, 1H). |
| 6 | 2′-NO₂, 5′-Cl | 25 | 130-134^{a} | C₁₉H₂₄ClN₃O₃.HC | 1340, 1520 | 1.17 (t, 3H); 1.50-1.70 (m, 1H); 1.70-1.95 (m, 4H); 2.23- |
| | | | | (C, H, N) | 1630 | 2.45 (m, 4H); 2.65-3.20 (m, 5H); 3.90 (br s, 1H); 4.72 (br, |
| | | | | | | s, 1H); 6.17-6.24 (dt, 1H); 7.05 (d, 1H); 7.30 (dd, 1H); |
| | | | | | | 7.56 (d, 1H); 7.92 (d, 1H). |
| 7 | 2′-Cl, 5′-NO₂ | 31 | 208-210^{a} | C₁₉H₂₄ClN₃O₃.HC | 1345, 1525 | 1.17 (t, 3H); 1.50-1.70 (m, 1H); 1.70-1.95 (m, 4H); 2.25- |
| | | | | (C, H, N) | 1640 | 2.45 (m, 4H); 2.80-3.20 (m, 5H); 3.95 (br s, 1H); 4.72 (br, |
| | | | | | | s, 1H); 6.34-6.48 (dt, 1H); 6.95 (d, 1H); 7.53 (d, 1H); 8.03 |
| | | | | | | (dd, 1H); 8.40 (d, 1H). |

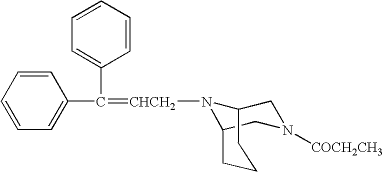

| | | Yield | m.p. | Formula | IR^{C} | ¹H-NMR |
|---|---|---|---|---|---|---|
| Ex. | R | % | ° C. | (Analysis^{b}) | ν cm⁻¹ | δ ppm |
| 15 | | 54 | 102-105^{a} | C₂₅H₃₀N₂.HCl | 1650 | 1.17 (t, 3H); 1.40-1.60 (m, 2H); 1.70-2.10 (m, 4H); 2.20- |
| | | | | (C, H, N) | | 2.40 (m, 2H); 2.89 (br s, 2H); 3.40-3.60 (m, 4H); 4.26 (d, |
| | | | | | | 2H); 6.18 (t, 1H); 7.00-7.50 (m, 10H). |

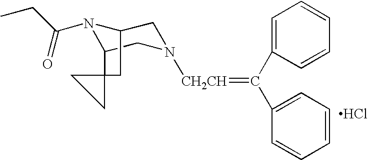

| Ex. | Yield % | m.p. ° C. |
|---|---|---|
| 16 | 59 | 55-57 |

General Procedure

A mixture of compounds (IVa) or (IVb) (2.30 mmol), the desired cinnamyl halide (2.30 mmol) and K₂CO₃ (2.30 mmol) in acetone or butanone (13.5 ml) was refluxed for 4-12 hours. Inorganic salts were filtered off, the filtrate was evaporated and the oily residue was purified by flash chromatography (eluent CH₂Cl₃: acetone/9:1) to give the compounds reported in the following tables as oils or as hydrochlorides.

### EXAMPLES 17-30

According to similar procedures, the following compounds were prepared:

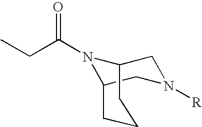

| Ex. | R | m.p. |
|---|---|---|
| 17 | 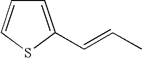 | 110° |
| 18 | 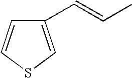 | 141° |
| 19 | 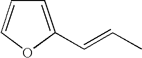 | 125-30° |
| 20 | 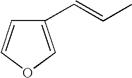 | 130-5° |
| 21 | 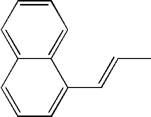 | oil |
| 22 | 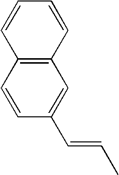 | oil |
| 23 | 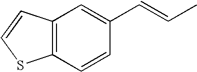 | 153° |
| Ex. | R | m.p. |
| 24 | 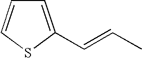 | 138° |
| 25 | 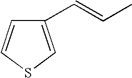 | 143° |
| 26 | 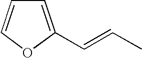 | 128-32° |
| 27 | 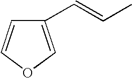 | 134-38° |
| 28 | 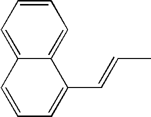 | oil |
| 29 | 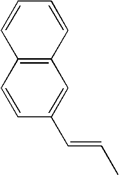 | oil |
| 30 | 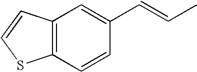 | 123-6° |

### EXAMPLE 31

Pharmacological Activity

Binding studies on the opioid receptors were carried out on mouse brain homogenates, in the presence of [³H]-DAMGO for μ [³H]-DELTORPHINE (II) for δ. [³H]-U69, 593 was used on guinea pigs homogenates to evaluate the κ binding. Morphine was used as the reference compound.

The results are reported in the following tables.

TABLE 1
Binding affinity to μ, δ and κ receptors

| | Binding affinities (Ki nM)^{a} | | |
|---|---|---|---|
| Compound of Ex. | μ | δ | κ |
| 1 | 29 ± 2.0 | 12000 ± 1152 | >50000 |
| 8 | 13 ± 1.5 | 1750 ± 144 | 2000 ± 180 |

^{a}Each value is the mean ± SEM of independent tests, each of them carried out in triplicate (n = 3).

TABLE 2
Inhibition constants towards μ opioid receptors

| | [³H]-DAMGO |
|---|---|
| Compound of Ex. | (Ki mM)^{a} |
| 2 | 29.0 |
| 3 | 70.0 |
| 4 | 48.33 |
| 8 | 13.0 |
| 9 | 7.66 |
| 10 | 8.66 |
| 11 | 5.83 |
| 12 | 18.0 |
| 13 | 6.0 |
| 14 | 6.0 |

^{a}Values of Ki were calculated based on K_{d} values of 1 nM for [³H]-DAMGO. Values are the mean from two experiments.

1. Compounds of formula 1: 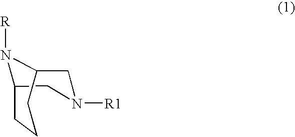
wherein
R is a group of formula 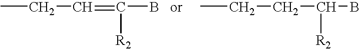
wherein
B is a C₆-C₁₀ aryl group, optionally substituted at the ortho-, meta- or para-positions with one or more substituents, which are the same or different, selected from the group consisting of C₁-C₃ alkoxy, C₁-C₂ halo alkyl, C₁-C₃ alkyl, halogens, carboxy, cyano, nitro; a C₅-C₇ cycloalkyl group, a 5 or 6 membered heterocyclic aromatic group, optionally benzofused, having at least one heteroatom selected from nitrogen, oxygen, sulfur; said heterocyclic group optionally having one or more substituents as described above for the aryl group;
R₂ is hydrogen, C₁-C₄ alkyl, C₅-C₇ cycloalkyl or a phenyl group optionally substituted as indicated above;
R₁ is a straight or branched C₂-C₈ acyl group
and the pharmaceutically acceptable salts thereof

2. Compounds as claimed in claim 1 wherein R is a group of formula 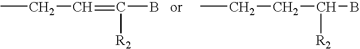
and B is an optionally substituted phenyl group as defined in claim 1, or a naphthyl group or a benzofused heterocyclic group.

3. Compounds as claimed in claim 1 wherein R is a group of formula

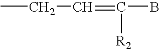

4. Compounds as claimed in claims 1-3 as central analgesic agents.

5. The use of the compounds of claims 1-3 for the preparation of analgesic medicaments.
